(19) United States
(12) Patent Application Publication
(10) Pub. No.: US 2026/0215687 A1
(43) Pub. Date: Jul. 30, 2026

(54) SYSTEMS AND METHODS FOR SUBJECT POSITIONING IN MAGNETIC RESONANCE IMAGING SYSTEMS

(71) Applicant: Hyperfine Operations, Inc., Guilford, CT (US)

(72) Inventors: Laura Sacolick, Guilford, CT (US); Michael Stephen Poole, Guilford, CT (US); Corinne Camay, Guilford, CT (US); Elena Gromova-Godoy, Guilford, CT (US)

(73) Assignee: Hyperfine Operations, Inc., Guilford, CT (US)

(21) Appl. No.: 19/370,703

(22) Filed: Oct. 27, 2025

Related U.S. Application Data
(63) Continuation of application No. PCT/US2024/026658, filed on Apr. 26, 2024.
(60) Provisional application No. 63/593,810, filed on Oct. 27, 2023.
(60) Provisional application No. 63/499,009, filed on Apr. 28, 2023.

(51) Int. Cl. A61B 5/00; A61B 5/055; G01R 33/44; G01R 33/56

(52) U.S. Cl. CPC A61B 5/0037; A61B 5/0013; A61B 5/055; A61B 5/7425; A61B 5/743; A61B 5/7475; G01R 33/445; G01R 33/5608

(57) ABSTRACT

Systems and methods for positioning subjects in magnetic resonance (MR) systems are disclosed. An MR system can capture preliminary imaging data indicative of a first position of a subject in an imaging system. The MR system can make a determination regarding the first position of the subject based on the preliminary imaging data and capture subsequent imaging data of the subject based on the determination. For example, positioning information can be provided automatically on a periodic and/or regular basis until a user indicates that positioning information is no longer desired.

### CROSS-REFERENCE TO RELATED APPLICATION

This application claims the benefit under 35 U.S.C. § 120 as a continuation of International Patent Application No. PCT/US2024/026658 filed April 26, 2024, which claims priority to U.S. Provisional Patent Application No. 63/593,810 filed October 27, 2023, and titled “Systems And Methods For Subject Positioning In Magnetic Resonance Imaging Systems,” and U.S. Provisional Patent Application No. 63/499,009 filed April 28, 2023, and titled “Improved Deep Learning-Based MR Reconstruction T2,” each of which is incorporated herein in its entirety.

### FIELD OF THE DISCLOSURE

The present disclosure relates generally to the field of subject positioning in magnetic resonance (MR) systems.

### BACKGROUND

Magnet resonance imaging (MRI) systems are used to generate images of the inside of the human body. MRI systems may be used to detect magnetic resonance (MR) signals in response to applied electromagnetic fields. However, capturing usable images of a subject (e.g., a patient) requires proper positioning of the subject within the MR system, which can be challenging to perform using manual processes alone.

### SUMMARY

Proper subject positioning within an MR system is a prerequisite to obtaining quality MR images. If the portion of the subject that is to be imaged is misaligned with the coil(s) of the MR system, the images produced by the MR system may include artifacts or may not fully capture the region of interest. In one non-limiting example, head position relative to a radio frequency (RF) coil affects how much of the head can be imaged. Poor head position can result in partial coverage of subject’s anatomy and reduced overall image quality overall. However, after the subject is placed into the RF coil, it is challenging to determine whether the subject is correctly positioned within the MR system to ensure optimal image quality.

The systems and methods described herein provide techniques for automatically determining the position of a subject within an MR system using fast, efficient position checks scans. The position check scans are used to obtain a single image of the subject in a brief period (e.g., one to two seconds). The image can be used to automatically determine the position of the subject relative to the coil(s) of the MR system, and whether the subject is to be repositioned within the MR system prior to performing an MR scan. In some implementations, the image is displayed with an indicator represents a volume over which the subsequent scans is to be acquired, to assist an operator with positioning the subject in the MR system. The position scans may be performed each time the subject is repositioned, or in some implementations, periodically (e.g., once every one to two seconds).

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects may be combined, and it will be readily appreciated that features described in the context of one aspect of the present disclosure may be combined with other aspects. Aspects may be implemented in any convenient form. In a non-limiting example, by appropriate computer programs, which may be carried on appropriate carrier media (computer readable media), which may be tangible carrier media (e.g., disks) or intangible carrier media (e.g., communications signals). Aspects may also be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIG. 1 illustrates example components of a magnetic resonance imaging system, which may be utilized to implement the techniques to perform positioning scans of subjects, in accordance with one or more implementations;

FIG. 2 illustrates an example flowchart of a first process for performing position check scans using an MR system, in accordance with one or more implementations;

FIG. 3 illustrates an example flowchart of a second process for performing continuous position check scans using an MR system, in accordance with one or more implementations;

FIG. 4 illustrates an example image generated using the techniques described herein including an indicator for positioning a subject, in accordance with one or more implementations;

FIG. 5 illustrates another example image generated using the techniques described herein, showing an amount by which a subject is misaligned with the MR system, in accordance with one or more implementations;

FIG. 6 illustrates an example image of a notification that may be displayed in response to detecting that a subject is misaligned with respect to the MR system, in accordance with one or more implementations;

FIG. 7 illustrates an example position check image displayed in connection with an indicator, in accordance with one or more implementations;

FIG. 8 illustrates an example position check image displayed in connection with an overlay indicator, in accordance with one or more implementations;

FIG. 9 illustrates an example position check image displayed in connection with indicators for an area to be imaged and an imageable area, in accordance with one or more implementations;

FIG. 10 illustrates an example position check image displayed in connection with an overlay indicator displayed within an area to be imaged and an imageable area, in accordance with one or more implementations;

FIG. 11 illustrates side-by-side comparisons of position check images displayed in connection with corresponding bounding-box indicators at various stages of alignment with the MR system, in accordance with one or more implementations;

FIG. 12 illustrates side-by-side comparisons of position check images displayed in connection with corresponding overlay indicators at various stages of alignment with the MR system, in accordance with one or more implementations;

FIGS. 13A – 13C depict example screenshots in which a patient head is not yet in the field of view (13A), the patient head is partially in the field of view (13B), and the patient head is within the field of view (13C), in accordance with one or more implementations;

FIG. 14 depicts an example screenshot of a view tab providing a live position check of a patient in the field of view, with the image in the field of view, in accordance with one or more implementations; and

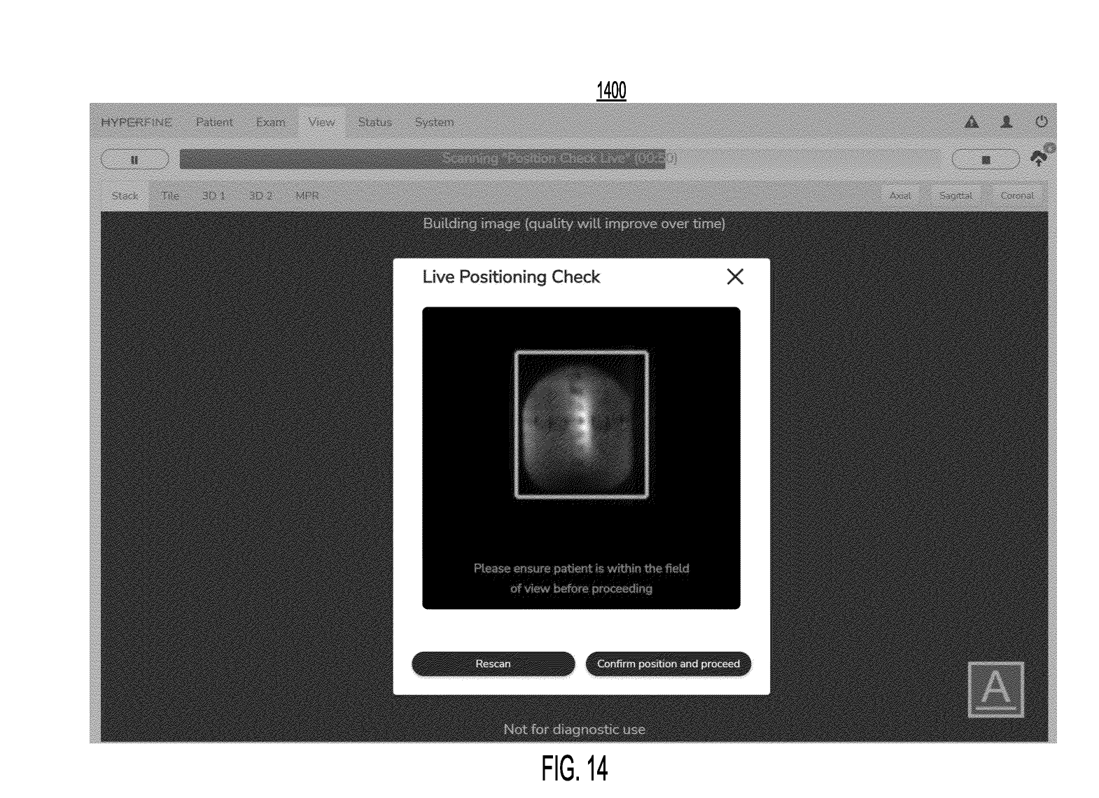

FIG. 15 is a block diagram of an example computing system suitable for use in the various arrangements described herein, in accordance with one or more example implementations.

### DETAILED DESCRIPTION

Below are detailed descriptions of various concepts related to and implementations of techniques, approaches, methods, apparatuses, and systems for removing electromagnetic interference from magnetic resonance images. The various concepts introduced above and discussed in detail below may be implemented in any of numerous ways, as the described concepts are not limited to any particular manner of implementation. Examples of specific implementations and applications are provided primarily for illustrative purposes.

Magnetic resonance imaging (MRI) systems use magnetic fields and electromagnetic signals to create detailed internal images of a subject in a non-invasive manner. In MR imaging, a magnetic field is applied to temporarily align protons in the subject, and one or more radiofrequency pulses are transmitted through the subject, which stimulate the protons and cause them to spin out of equilibrium. As the protons return to their original positions, they transmit radio signals that are detected by corresponding receive coils of the MRI systems. The received signals encode information about the structure and delineation of different tissues within the subject and can be used to reconstruct two-dimensional (2D) or three-dimensional (3D) internal images using image reconstruction techniques.

To properly detect signals emitted by the protons within a subject during an MRI scan, the subject must be positioned correctly relative to the receivers of the MR system. Otherwise, images produced by the MR system may include artifacts or may not fully capture a desired region of the subject. To ensure proper positioning of the subject prior to performing an MR scan, the MR system can perform a position check scan and determine whether the subject is positioned properly with respect to, in a non-limiting example, the RF coil(s) of the MR system. In one non-limiting example, the position check scan can generate a single 2D projection image (sometimes referred to herein as a “position check image”). The position check image can be displayed with an indicator, such as a bounding box or an overlay, which represents a region at which the subsequent scans will be acquired. An operator of the MR system can reposition the subject as needed, any may perform additional position check scans to verify that the subject is properly positioned within the system before proceeding with a full MR scan. In example embodiments, a “faster” scan or determination (e.g., one that takes a relatively short time) can be used to assess a subject’s position prior to a “slower” scan, in MRI systems or in other imaging modalities.

Various embodiments of the disclosed systems and methods can relate to a live patient positioning feature that allows the user/operator to see visual feedback in the device’s user interface of the patient’s position in comparison to the field of view. By showing the user live visual feedback, the user is able to reposition and adjust the patient to optimize the position faster. The visualization can be provided on the same tab/window as the exam list so the user does not need to flip between pages to see the image. The live patient positioning feature can allow the user to see the position of the patient as the patient moves into the system versus needing to stop and check the feedback and then readjust. This improvement in workflow can allow for a substantial time savings and optimal use of hospital staffing resources. This improvement can encourage the user to be looking at the visual by putting it on the same tab as the exam list. Previously, users would potentially need to go back and forth between positioning and verifying. This is further improved by the condensing of the exam and the view tab so the user is not flipping between these tabs to verify the patient’s position in the field of view.

The system can provide improved patient positioning, improved speed to position the patient, and optimized use of staffing resources needed to load the patient. The live patient positioning feature can be helpful for difficult to position patients, such as patients with a breathing tube or larger patients. In a usability evaluation, this feature saved 39 seconds on average in the time taken to position the patient across respective users compared to snap shot imaging after loading. Users without the condensed view were shown to frequently forget to check that the patient was properly positioned in the field of view.

In various embodiments, the live positioning pulse sequence can be based on a single shot fast spin echo acquisition which is (e.g., automatically scheduled to be) repeated in a loop until it is stopped. 3D volume is excited, but a 2D image is acquired. The image represents projection in the second phase-encode dimension. For example, if imaging is performed in coronal scan plane, the resulting image would look like all coronal slices of the head were summed together. The acquired images can be automatically updated to the visual in the tab, e.g., to provide near real-time video updates to facilitate positioning. The time between repetitions is controlled by a parameter called repetition time. Example embodiments of this method can be performed in any scan plane, with or without fat suppression. Each acquisition and reconstruction can be performed in a short time (e.g., less than 2 seconds, less than 1 second, less than 0.5 seconds, etc.).

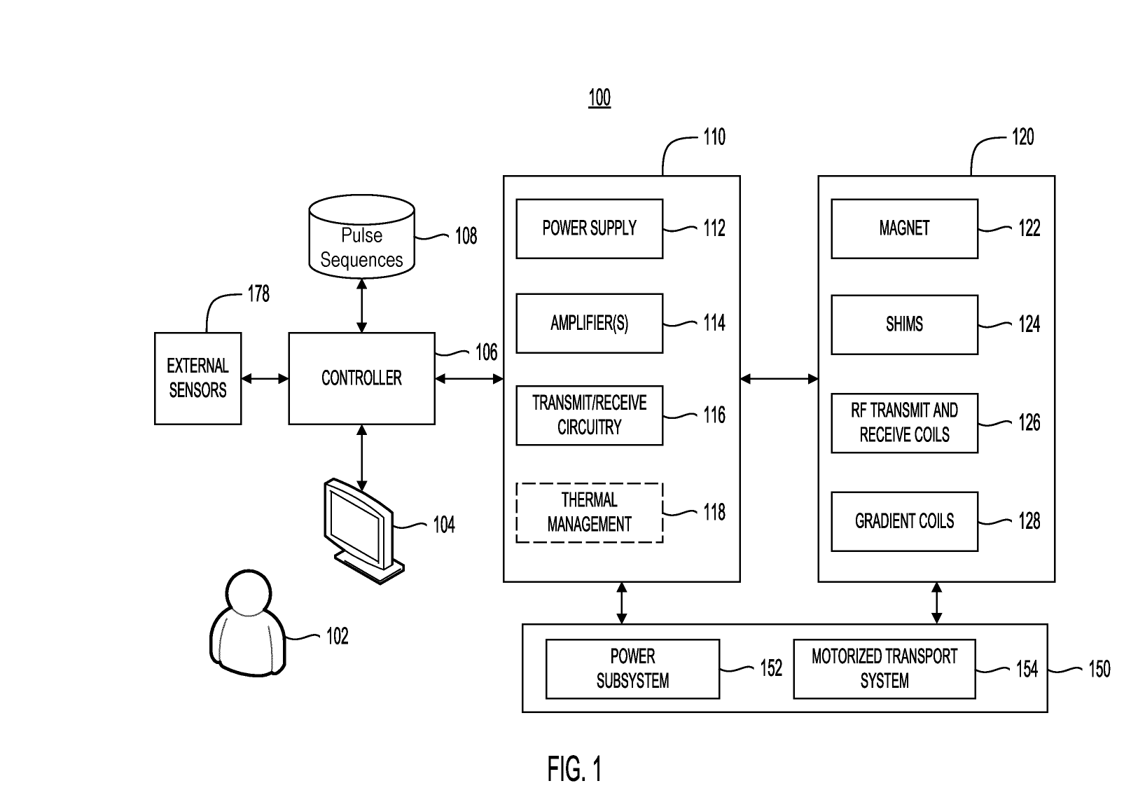

FIG. 1 illustrates an example MRI system which may be utilized in connection with the subject position scan techniques described herein. In FIG. 1, MRI system 100 may include a computing device 104, a controller 106, a pulse sequences repository 108, a power management system 110, and magnetics components 120. The MRI system 100 is illustrative, and an MRI system may have one or more other components of any suitable type in addition to or instead of the components illustrated in FIG. 1. Additionally, the implementation of components for a particular MRI system may differ from those described herein. Examples of low-field MRI systems may include portable MRI systems, which may have a field strength that may be, in a non-limiting example, less than or equal to 0.5 T, that may be less than or equal to 0.2 T, that may be within a range from 1 mT to 100 mT, that may be within a range from 50 mT to 0.1 T, that may be within a range of 40 mT to 80 mT, that may be about 64 mT, etc.

The magnetics components 120 may include B₀ magnets 122, shims 124, RF transmit and receive coils 126, and gradient coils 128. The B₀ magnets 122 may be used to generate a main magnetic field B₀. B₀ magnets 122 may be any suitable type or combination of magnetics components that may generate a desired main magnetic B₀ field. In some embodiments, B₀ magnets 122 may be one or more permanent magnets, one or more electromagnets, one or more superconducting magnets, or a hybrid magnet comprising one or more permanent magnets and one or more electromagnets or one or more superconducting magnets. In some embodiments, B₀ magnets 122 may be configured to generate a B₀ magnetic field having a field strength that may be less than or equal to 0.2 T or within a range from 50 mT to 0.1 T.

In some implementations, the B₀ magnets 122 may include a first and second B₀ magnet, which may each include permanent magnet blocks arranged in concentric rings about a common center. The first and second B₀ magnet may be arranged in a bi-planar configuration such that the imaging region is located between the first and second B₀ magnets. In some embodiments, the first and second B₀ magnets may each be coupled to and supported by a ferromagnetic yoke configured to capture and direct magnetic flux from the first and second B₀ magnets.

MRI scans are performed by exciting and detecting emitted MR signals using transmit and receive coils 126, respectively (referred to herein as RF coils). The transmit and receive coils 126 can include separate coils for transmitting and receiving, multiple coils for transmitting or receiving, or the same coils for transmitting and receiving. Thus, a transmit/receive component may include one or more coils for transmitting, one or more coils for receiving, or one or more coils for transmitting and receiving. The transmit/receive coils may be referred to as Tx/Rx or Tx/Rx coils to generically refer to the various configurations for transmit and receive magnetics components of an MRI system. These terms are used interchangeably herein. In FIG. 1, RF transmit and receive coils 126 may include one or more transmit coils that may be used to generate RF pulses to induce an oscillating magnetic field B₁. The transmit coil(s) may be configured to generate any type of suitable RF pulses.

The power management system 110 includes electronics to provide operating power to one or more components of the MRI system 100. In a non-limiting example, the power management system 110 may include one or more power supplies, energy storage devices, gradient power components, transmit coil components, or any other suitable power electronics needed to provide suitable operating power to energize and operate components of MRI system 100. As illustrated in FIG. 1, the power management system 110 may include a power supply system 112, power component(s) 114, transmit/receive circuitry 116, and may optionally include thermal management components 118 (e.g., cryogenic cooling equipment for superconducting magnets, water cooling equipment for electromagnets).

The power supply system 112 may include electronics that provide operating power to magnetic components 120 of the MRI system 100. The electronics of the power supply system 112 may provide, in a non-limiting example, operating power to one or more gradient coils (e.g., gradient coils 128) to generate one or more gradient magnetic fields to provide spatial encoding of the MR signals. Additionally, the electronics of the power supply system 112 may provide operating power to one or more RF coils (e.g., RF transmit and receive coils 126) to generate or receive one or more RF signals from the subject or EMI signals from the environment.

In a non-limiting example, the power supply system 112 may include a power supply configured to provide power from mains electricity to the MRI system or an energy storage device. The power supply may, in some embodiments, be an AC-to-DC power supply that converts AC power from mains electricity into DC power for use by the MRI system. The energy storage device may, in some embodiments, be any one of a battery, a capacitor, an ultracapacitor, a flywheel, or any other suitable energy storage apparatus that may bi-directionally receive (e.g., store) power from mains electricity and supply power to the MRI system. Additionally, the power supply system 112 may include additional power electronics including, but not limited to, power converters, switches, buses, drivers, and any other suitable electronics for supplying the MRI system with power.

The amplifiers(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and may provide power to one or more shim power components configured to provide power to one or more shims (e.g., shims 124). In some implementations, the shims 124 may be implemented using permanent magnets, electromagnetics (e.g., a coil), or combinations thereof. The transmit/receive circuitry 116 may be used to select whether RF transmit coils or RF receive coils 126 are being operated.

As illustrated in FIG. 1, the MRI system 100 may include the controller 106 (also referred to as a console), which may include control electronics to send instructions to and receive information from power management system 110. The controller 106 may be configured to implement one or more pulse sequences, which are used to determine the instructions sent to power management system 110 to operate the magnetic components 120 in a desired sequence (e.g., parameters for operating the RF transmit and receive coils 126, parameters for operating gradient coils 128, etc.). Additionally, the controller 106 may execute processes to remove electromagnetic interference from magnetic resonance images according to various techniques described herein. A pulse sequence may generally describe the order and timing in which the RF transmit and receive coils 126 and the gradient coils 128 operate to acquire resulting MR data. In a non-limiting example, a pulse sequence may indicate an order and duration of transmit pulses, gradient pulses, and acquisition times during which the receive coils acquire MR data.

A pulse sequence (e.g., stored in the pulse sequence repository 108) may be organized into a series of periods. In a non-limiting example, a pulse sequence may include a pre-programmed number of pulse repetition periods, and applying a pulse sequence may include operating the MRI system in accordance with parameters of the pulse sequence for the pre-programmed number of pulse repetition periods. In each period, the pulse sequence may include parameters for generating RF pulses (e.g., parameters identifying transmit duration, waveform, amplitude, phase, etc.), parameters for generating gradient fields (e.g., parameters identifying transmit duration, waveform, amplitude, phase, etc.), timing parameters governing when RF or gradient pulses are generated or when the receive coil(s) are configured to detect MR signals generated by the subject, among other functionality.

Examples of pulse sequences include zero echo time (ZTE) pulse sequences, balance steady-state free precession (bSSFP) pulse sequences, gradient echo pulse sequences, inversion recovery pulse sequences, FLAIR pulse sequences, DWI pulse sequences, spin echo pulse sequences including conventional spin echo (CSE) pulse sequences, multi-shot fast-spin echo (FSE) pulse sequences, turbo spin echo (TSE) pulse sequences or any multi-spin echo pulse sequences such a diffusion weighted spin echo pulse sequences, inversion recovery spin echo pulse sequences, arterial spin labeling pulse sequences, and Overhauser imaging pulse sequences, among others.

As illustrated in FIG. 1, the controller 106 may communicate with the computing device 104, which may be programmed to process received MR data (e.g., the scan data produced via the position scans described herein). In a non-limiting example, the computing device 104 may process received MR data to generate one or more MR images using any suitable image reconstruction processes. Additionally or alternatively, the controller 106 may process received MR data to perform image reconstruction, and the reconstructed image may be provided to the computing device 104 for display. The controller 106 may provide information about one or more pulse sequences to computing device 104 for the processing of data by the computing device.

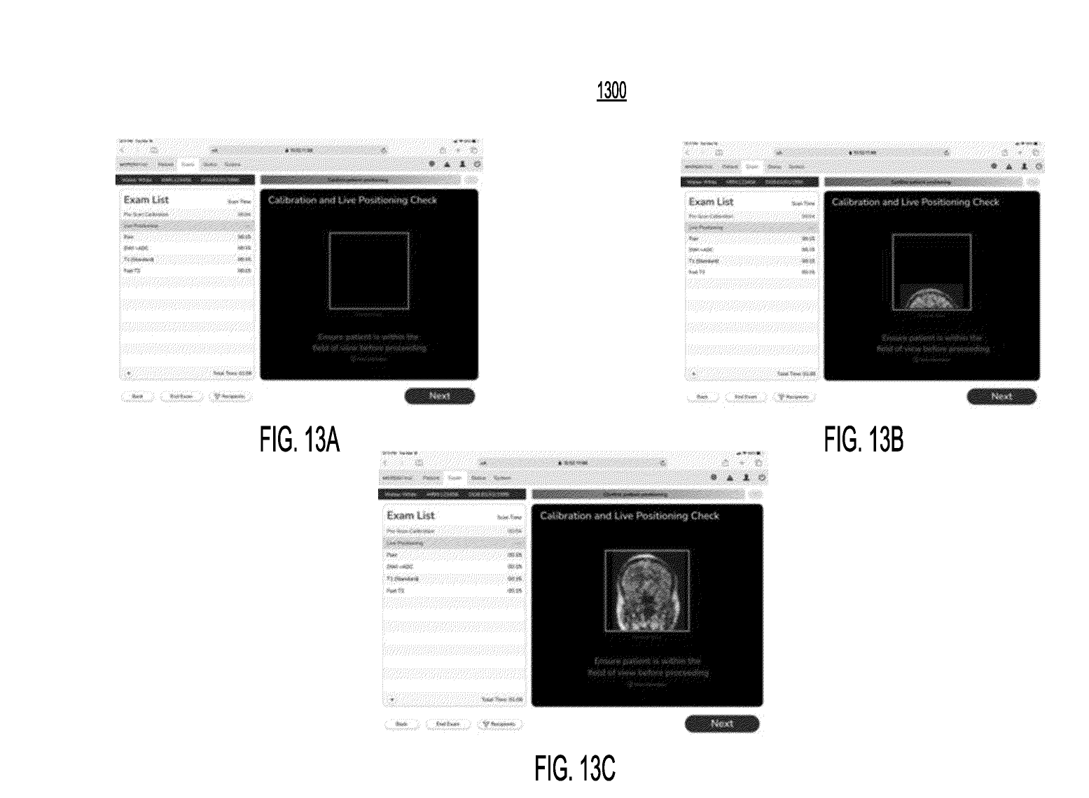

The computing device 104 may be any electronic device configured to process acquired MR data and generate one or more images of a subject being imaged. The computing device 104 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), a tensor processing unity (TPU), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor may read instructions. The instructions may include code generated from any suitable computer programming language. The computing device 104 may include any or all of the components and perform any or all of the functions of the computer system 1300 described in connection with FIG. 13. In some implementations, the computing device 104 may be located in a same room as the MRI system 100 or coupled to the MRI system 100 via wired or wireless connection.

In some implementations, computing device 104 may be a fixed electronic device such as a desktop computer, a server, a rack-mounted computer, or any other suitable fixed electronic device that may be configured to process MR data and generate one or more images from MR signals captured using the MRI system 100. Alternatively, computing device 104 may be a portable device such as a smart phone, a personal digital assistant, a laptop computer, a tablet computer, or any other portable device that may be configured to process MR signal data (e.g., perform image reconstruction, other processing operations, etc.). In some implementations, computing device 104 may comprise multiple computing devices of any suitable type, as aspects of the disclosure provided herein are not limited in this respect. In some implementations, operations that are described as being performed by the computing device 104 may instead be performed by the controller 106, or vice-versa. In some implementations, certain operations may be performed by both the controller 106 and the computing device 104 via communications between said devices.

The computing device 104 may include or may be in communication with one or more displays, audio devices (e.g., speakers, alarms, etc.), haptic devices, or other output devices that can provide output to an operator of the MR system. Output can be provided to indicate that a subject is properly or improperly positioned within the MR system 100. The output may include one or more indicators that show or otherwise indicate a relative position of the subject within the MR system (e.g., a distance from a one of the RF coils 126, a field-of-view (FOV) of the MR system, etc.).

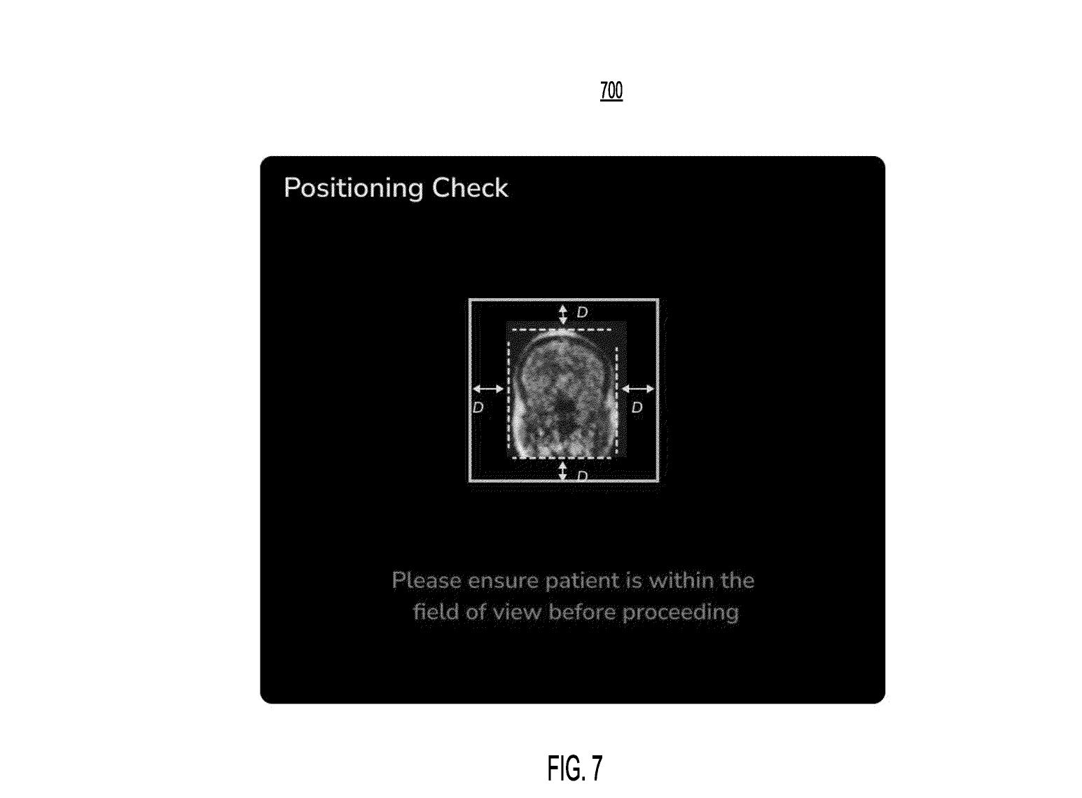

The output may include audio alarms or audio notifications that indicates whether the subject correctly positioned within the MR system. The output may include haptic feedback that indicates whether the subject correctly positioned within the MR system. Position scan images captured using the techniques described herein may be displayed to the operator with one or more overlays or bounding boxes that represent an imageable area of the MR system (e.g., the FOV of the MR system), an area to be imaged by the MR system, or a relative position of the subject within the MR system, among others, as shown in FIGS. 7-12.

The MRI system 100 may include one or more external sensors 178. The one or more external sensors may assist in detecting one or more error sources (e.g., motion, noise) which degrade image quality. The controller 106 may be configured to receive information from the one or more external sensors 178. In some embodiments, the controller 106 of the MRI system 100 may be configured to control operations of the one or more external sensors 178, as well as collect information from the one or more external sensors 178. The data collected from the one or more external sensors 178 may be stored in a suitable computer memory and may be utilized to assist with various processing operations of the MRI system 100.

The MRI system 100 may be a portable MRI system, and therefore may include portable subsystems 150. The portable subsystems 150 may include at least one power subsystem 152 and at least one motorized transport system 154. The power subsystem 152 may include any device or system that enables or supports the portability of the MRI system 100. In a non-limiting example, the power subsystem 152 may include any of the functionality of the power supply 112, and may further include other circuitry enabling the provision of electric power, including but not limited to as batteries and associated circuitry, AC-DC converters, DC-DC converters, switching power converters, voltage regulators, or battery charging circuitry, among others. The power subsystem 152 may include connectors that support the portability of the MRI system 100, such as connectors and cables of a suitable size for a portable system. In some implementations, the power subsystem 152 may include circuitry that provides power to the MRI system 100. In some implementations, the power subsystem 152 may include circuitry or connectors that enable the MRI system 100 to receive power from one or more power outlets, which may include standard power outlets.

The motorized transport system 154 can include any device or system that allows the MRI system 100 to be transported to different locations. The motorized transport system 154 may include one or more components configured to facilitate movement of the MRI system 100 to a location at which MRI is needed. In some implementations, the motorized transport system 154 may include a motor coupled to drive wheels. In such implementations, the motorized transport system 154 may provide motorized assistance in transporting MRI system 100 to one or more locations. The motorized transport system 154 may include a plurality of castors to assist with support and stability as well as facilitating transport.

In some implementations, the motorized transport system 154 includes motorized assistance controlled using a controller (e.g., a joystick or other controller that can be manipulated by a person) to guide the portable MRI system during transportation to desired locations. The motorized transport system 154 may include power assist circuitry (e.g., including accelerometers and vibration sensors, etc.) that detects when force is applied to the MRI system and, in response, engages the motorized transport system 154 to provide motorized assistance in the direction of the detected force. In some implementations, the motorized transport system 154 can detect when force is applied to one or more portions of the MRI system 100 (e.g., by an operator pushing on or applying force to a rail, housing, etc., of the MRI system 100) and, in response, provide motorized assistance to drive the wheels in the direction of the applied force. The MRI system 100 can therefore be guided to locations where MRI is needed. The power subsystem 152 can be utilized to provide power to the MRI system 100, including the motorized transport system 154.

In some implementations, the motorized transport system 154 may include a safety mechanism that detects collisions. In a non-limiting example, the motorized transport system 154 can include one or more sensors that detect the force of contact with another object (e.g., a wall, bed, or other structure). Upon detection of a collision, the motorized transport system 154 can generate a signal to one or more motors or actuators of the motorized transport system 154, to cause a motorized locomotion response away from the source of the collision. In some implementations, the MRI system 100 may be transported by having personnel move the system to desired locations using manual force. In such implementations, the motorized transport system 154 may include wheels, bearings, or other mechanical devices that enable the MRI system 100 to be repositioned using manual force.

As described herein, the controller 106 and/or the computing device 104 may include one or more processors and memory, and may include any or all of the components and perform any or all of the functions of the computer system 1300 described in connection with FIG. 13. The controller 106 or the computer 104 may control one or more of the components of the MRI system 100 to perform the subject positioning scan techniques described herein.

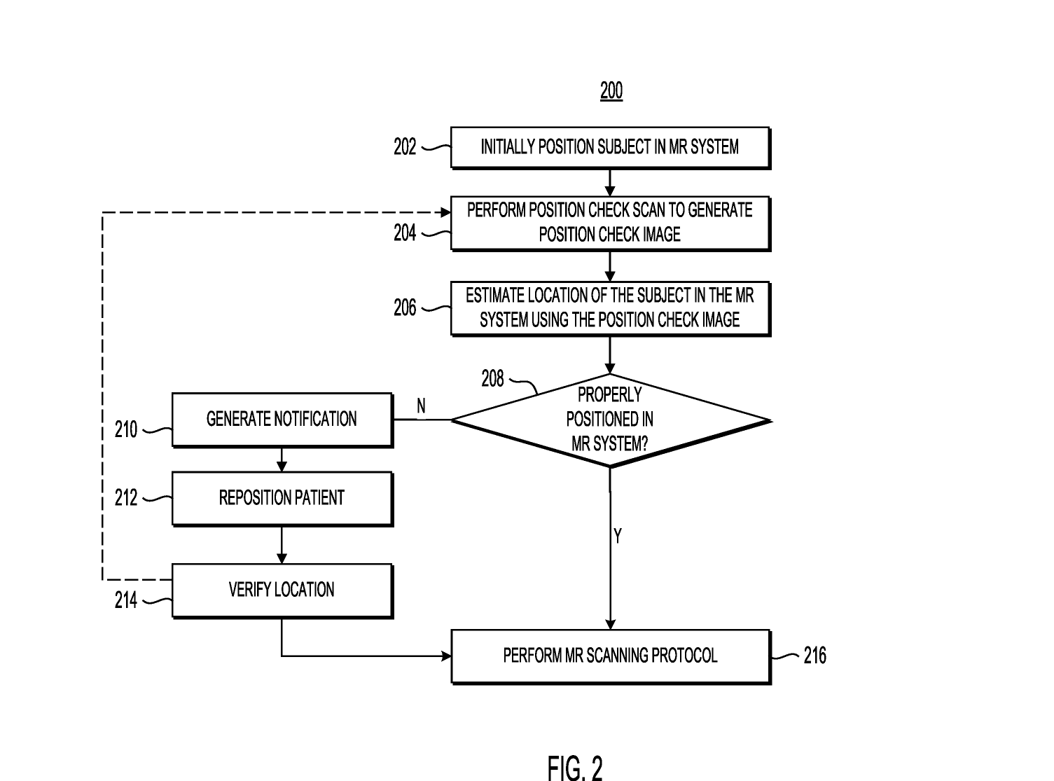

Referring to FIG. 2, illustrated is an example flowchart of a first process 200 for performing position check scans using an MR system (e.g., the MR system 100), in accordance with one or more implementations. The process 200 may be executed using any suitable computing system (e.g., the controller 106, the computing device 104 of FIG. 1, the computing system 1300 of FIG. 13, etc.) of an MR system (e.g., the MRI system 100). The MR system used to perform the process 200 may be a low-field MR system. The MR system used to perform the process 200 may have a magnetic field inhomogeneity of less than or equal to 1000 parts-per-million. The MR system used to perform the process 200 may have a magnetic resonance frequency inhomogeneity greater than 1000 Hz. It may be appreciated that certain steps of the process 200 may be executed in parallel (e.g., concurrently) or sequentially, while still achieving useful results.

At step 202, the subject can be initially positioned within the MR system, for example, by way of assistance from an operator of the MR system. Due to the configuration of the components of the MR system, and variations in different subject anatomy, manually positioning the subject may not correctly align the subject (e.g., a region of interest (ROI) of the subject) with the RF coils of the MR system. Positioning the subject may include inserting a ROI of the subject into an enclosure of the MR system. In a non-limiting example, the ROI may be the head of the subject.

At step 204, the operator can interact with the MR system to cause the MR system to perform a position check scan, which can capture at least a portion of the ROI of the subject to be imaged. In some implementations, calibration scans or processes may be performed prior to performing the position check scan. The position check scan can be a scan that generates a 2D projection image of the ROI of the subject. In some implementations, the 2D projection can be captured such that skin, bone, or other outer tissue is not depicted in the image. In a non-limiting example, if the ROI of the subject is the head of the subject, the 2D projection image may depict internal soft tissue such as the brain of the patient. The data captured by the MR system for the position check scan is processed using image reconstruction techniques to generate the 2D projection image.

The position check scan may be any type of scan that is capable of capturing a view of the subject within a suitable period of time (e.g., one to two seconds, etc.). In some implementations, the position check scan may be performed using a scan protocol, such as fast-spin echo (FSE), echo planar imaging (EPI), to acquire raw MRI data (e.g., k-space data). In some implementations, a particular sequencing process may be applied during the position check scan to remove portions of the MR signal that may obscure tissues of interest. In a non-limiting example, the sequencing process may implement a signal suppression technique, such as short inverse recovery, that suppresses fat signals. In some implementations, additional or alternative signal separation techniques may be utilized, such as spectrally selective RF pulses, or Dixon-type fat-water separation.

MR signal data (e.g., frequency domain data) captured using the position check scan can be processed using image reconstruction techniques to generate a position check image. The position check image may be indicative of a location of the ROI of the subject within the MR system. Non-limiting examples of position check images are shown in FIGS. 7-12. In some implementations, multiple images may be constructed from the position check scan. In a non-limiting example, images from different perspectives (e.g., multiplanar images) may be captured for display to an operator of the MR system, to facilitate proper positioning of a patient.

At step 206, the MR system can estimate a location of the subject in the MR system using the position check image. In a non-limiting example, the MR system may utilize empirical or rule-based techniques to determine a distance of the anatomy of the patient depicted in the position check image from a desired location within the MR system. Furthering this example, the MR system may determine a distance between one or more boundaries of the subject’s anatomy (e.g., the ROI) as depicted in the position check image from a corresponding predetermined boundaries of the image, to estimate a relative distance between the subject and the RF coils (e.g., the coils 126) of the MR system.

In some implementations, trained machine-learning models, such as convolutional neural networks (CNNs) can be utilized to determine the distance between the patient and a desired position within the MR system. In a non-limiting example, the machine-learning models can be trained using supervised learning, semi-supervised learning, or self-supervised learning using training datasets including position check images with known distances between the patient and the RF coils of the MR system used to captured said position check images. The known distances can be used as labels or the training process. In such implementations, the position check image can be provided as input to the trained machine-learning model, which can be executed to generate the distance and/or relative location of the subject within the MR system.

At step 208, upon estimating the relative location of the subject, the MR system can compare the relative location to one or more thresholds or boundaries to determine whether the patient is to be repositioned within the MR system. For example, the MR system can compare the distance between the ROI of the patient and the RF coils of the MR system to determine whether the subject is within a threshold distance of the RF coils. In some implementations, the MR system may compare the relative location of the ROI of the patient to an imageable area of the MR system to determine whether the entirety of the ROI is positioned within the imageable area. If the ROI of the subject is within the imageable area and/or within a threshold distance of the RF coils of the MR system, the MR system can proceed to step 216. If the ROI of the subject is not entirely within the imageable area and/or not within a threshold distance of the RF coils of the MR system, the MR system can proceed to step 210.

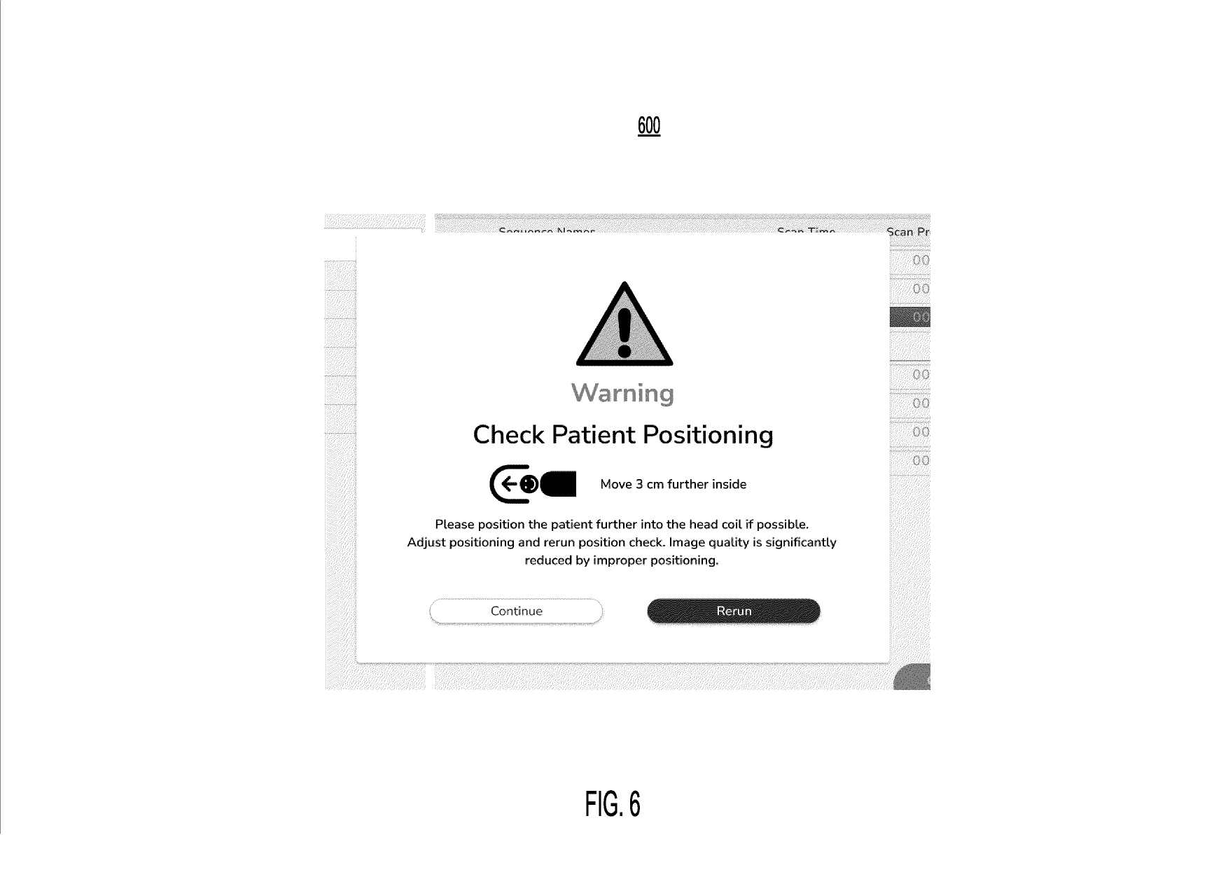

At step 210, the MR system can generate a notification for the operator to indicate that the subject is to be repositioned in the MR system. The notification may be or include an indicator light, a graphical user interface, audio feedback, haptic feedback, other types of feedback or guidance information/signal, or combinations thereof. In an implementation where the notification is displayed as a graphical user interface, the notification may include an indication of the estimated location of the subject in the MR system (e.g., a distance from the ROI of the subject from the RF coil, etc.). In a non-limiting example, the notification may include a distance between the top of the subject’s head/skull/brain and one or more RF coils of the MR system. One example of a notification is shown in FIG. 6.

Referring now to FIG. 6 illustrated is an example image 400 generated using the techniques described herein including an indicator for positioning a subject, in accordance with one or more implementations. As shown in FIG. 6, a graphical user interface is generated and displayed to the operator of the MR system. The notification includes instructions to reposition the subject. The notification includes an indication of a distance to move the subject (e.g., 3cm further inside). The notification includes interactive buttons that enable the operator to rerun a position check scan or to continue to run a full MR scan.

Referring back to FIG. 2, at step 212, the operator of the MR system can reposition the subject according to the notification generated by the MR system. Repositioning the patient may include moving the ROI of the subject within as indicated in the notification. Repositioning the subject may include moving the ROI to be closer to the RF coils of the MR system. Once the subject has been moved, the operator can interact with the MR system to proceed with a full MR scan (e.g., execute step 216), or to verify the position of the patient at step 214.

At step 214, the MR system can verify the position of the subject after the subject has been moved in step 212. In a non-limiting example, the operator may provide input to re-execute the position check scan to verify that the subject’s new position is suitable for a full MR scan. To verify the position, the MR system can re-execute steps 204-210 of the process 200 to perform additional position check scans, estimate corresponding locations of the subject within the MR system, and determine whether the subject is properly positioned and/or aligned within the MR system as described herein. If the subject is still not positioned and/or aligned correctly in the MR system, the operator can repeat the process of repositioning, re-alignment and/or performing position check scans to verify that the patient is positioned and/or aligned correctly in the MR system.

At step 216, the MR system can proceed to perform a full scan of the ROI of the subject. At this stage of the process, the subject is correctly positioned and/or aligned within the MR system and the MR system proceeds to perform a full scan of the ROI of the patient. Any suitable scan can be performed by the MR system. The scans may include but are not limited to T1, T2, FLAIR, or DWI scans.

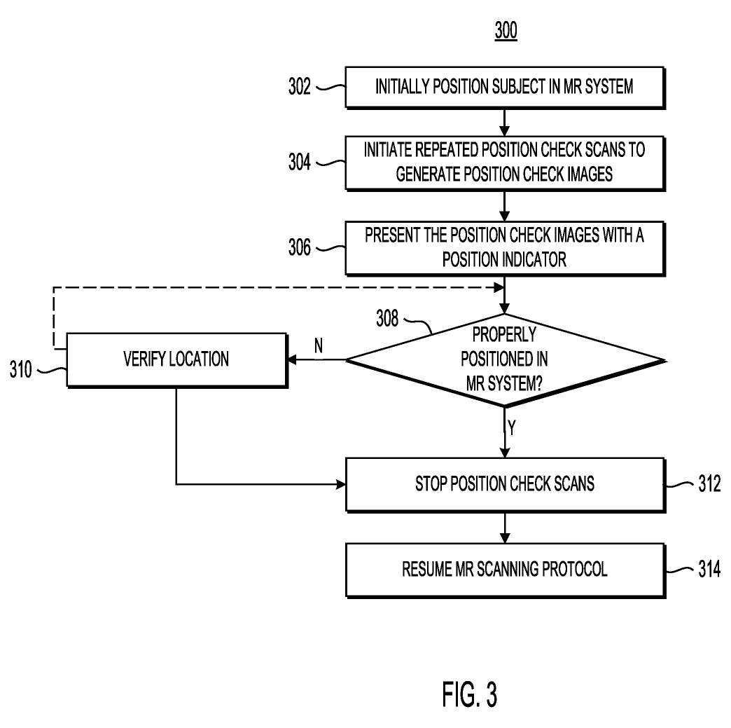

Referring to FIG. 3, illustrated is an example flowchart of a second process 300 for performing continuous (or live/dynamic) position check scans using an MR system, in accordance with one or more implementations. The process 300 may be executed using any suitable computing system (e.g., the controller 106, the computing device 104 of FIG. 1, the computing system 1300 of FIG. 13, etc.) of an MR system (e.g., the MRI system 100). The MR system used to perform the process 300 may be a low-field MR system. The MR system used to perform the process 300 may have a magnetic field inhomogeneity of less than or equal to 1000 parts-per-million. The MR system used to perform the process 300 may have a magnetic resonance frequency inhomogeneity greater than 1000 Hz. It may be appreciated that certain steps of the process 300 may be executed in parallel (e.g., concurrently) or sequentially, while still achieving useful results.

The process 300 may be similar to the process 200 of FIG. 2, with position check scans of the subject being periodically performed in a continuous fashion until explicitly halted by the operator of the MR system. In some implementations, the position check scans can be automatically terminated when the subject is correctly positioned within the MR system.

At step 302, the subject can be initially positioned within the MR system, for example, by way of assistance from an operator of the MR system. Due to the configuration of the components of the MR system, and variations in different subject anatomy, manually positioning the subject (e.g., via visual assessment) may not correctly align the subject (e.g., an ROI of the subject) with respect to the RF coils of the MR system. Positioning the subject may include inserting/locating/moving/maneuvering a ROI of the subject into an enclosure of the MR system. In a non-limiting example, the ROI may be the head/brain of the subject.

At step 304, the operator can interact with the MR system to cause the MR system to initiated repeated position check scans of the patient. As described herein, position check scans capture at least a portion of the ROI of the subject to be imaged when the subject has been initially positioned in the MR system. In some implementations, calibration scans or processes may be performed prior to initiating the repeated position check scans. Each position check scan can generate a 2D projection image (or 1-D measurement, or other 1-D/2-D/3-D measurement or representation) of the ROI of the subject, as described herein. The repeated/continuous position check scans can be performed as the patient is moved relative to the coils or imaging region of the MR system. For instance, the MR system can perform calibration, perform scanning and/or produce an image at intervals, e.g., at 1- second intervals. Some or all of these steps/operations may be performed sequentially or in parallel, in a loop and/or according to a defined sequence/order. In a non-limiting example, if the ROI of the subject is the head of the subject, the 2D projection image may depict internal soft tissue such as the brain of the patient. The data captured by the MR system for the position check scan is processed using image reconstruction techniques to generate the 2D projection image. As described herein, each position check scan may be an FSE scan, or other types of scans such as EPI or fast GRE. Signal suppression techniques, such as short inverse recovery, may be used to suppress fat signals in the MR signal. In some implementations, additional or alternative signal separation techniques may be utilized, such as spectrally selective RF pulses, or Dixon-type fat-water separation.

At step 306, the position check images can be presented to the operator of the MR system (e.g., in real time or near real time, as the patient is moved/adjusted relative to the coils / MR system). The position check images can be presented, in a non-limiting example, in connection with one or more indictors. The indicators may include indicators for an imageable area of the MR system, indicators for an area to be imaged by the MR system, and/or indicators corresponding to whether the subject is correctly positioned with respect to the MR system, as described herein. In a non-limiting example, the MR system may utilize empirical or rule-based techniques to determine a distance of the anatomy of the patient depicted in the position check image.

The MR system may determine a distance between one or more boundaries of the subject’s anatomy (e.g., the ROI) as depicted in the position check image from a corresponding predetermined boundaries of the image, to estimate a relative distance between the subject and the RF coils (e.g., the coils 126) of the MR system. In some implementations, trained machine-learning models, such as CNNs can be utilized to determine the distance between the patient and a desired position within the MR system, as described herein. The MR system can determine the distance for each position check image generated by the system. As described herein, once initialized, position check scans can be performed repeatedly to generate position check images at a periodic rate (e.g., one every two seconds, etc.). The MR system can estimate the location of the patient, and present the position check image (with any indicators, e.g., as feedback or guidance) to the operator as each image is generated.

At step 308, the MR system can compare the relative location of the subject to one or more thresholds or boundaries to determine whether the patient is to be repositioned within the MR system. For example, the MR system can compare the distance between the ROI of the patient and the RF coils of the MR system to determine whether the subject is within a threshold distance of the RF coils. In some implementations, the MR system may compare the relative location of the ROI of the patient to an imageable area of the MR system to determine whether the entirety of the ROI is positioned within the imageable area. If the ROI of the subject is within the imageable area and/or within a threshold distance of the RF coils of the MR system, the MR system can proceed to step 312. If the ROI of the subject is not entirely within the imageable area and/or not within a threshold distance of the RF coils of the MR system, the MR system can proceed to step 310. In some implementations, the indicators displayed in connection with the position check image(s) may be updated to reflect the degree to which the ROI of the subject is properly positioned within the MR system. Examples of said indicators are shown in FIGS. 7-12.

At step 310, the operator of the MR system can observe the position check images while repositioning the patient to verify whether the patient is properly positioned and/or aligned within the MR system. For example, one or more indicators, as described herein, may indicate the degree to which the patient is to be repositioned in the MR system. The operator can reposition/adjust/move the patient and observe the change as captured by the MR system as further position check images are continuously generated. Once the subject has been properly positioned within the MR system, the MR system can proceed to execute step 312.

At step 312, the MR system can terminate the continuous process of capturing position check images of the subject. In one implementation, the operator of the MR system may provide input to cease the continuous capturing of position check images once the subject has been properly positioned. In another implementation, the MR may automatically terminate capturing of position check images upon detecting that the subject is properly positioned within the MR system relative to the RF coils.

At step 314, once the MR system has stopped performing position check scans (e.g., in response to achieving suitable/acceptable positioning and/or alignment of the subject, or in response to receiving an indication/confirmation from the MR system or an operator), the MR system can perform a full scan of the ROI of the subject. As described herein, at this stage of the process, the subject is correctly positioned and/or aligned within the MR system, and the MR system can proceed to perform a full scan of the ROI of the patient. Any suitable scan can be performed by the MR system. The scans may include but are not limited to T1, T2, FLAIR, or DWI scans.

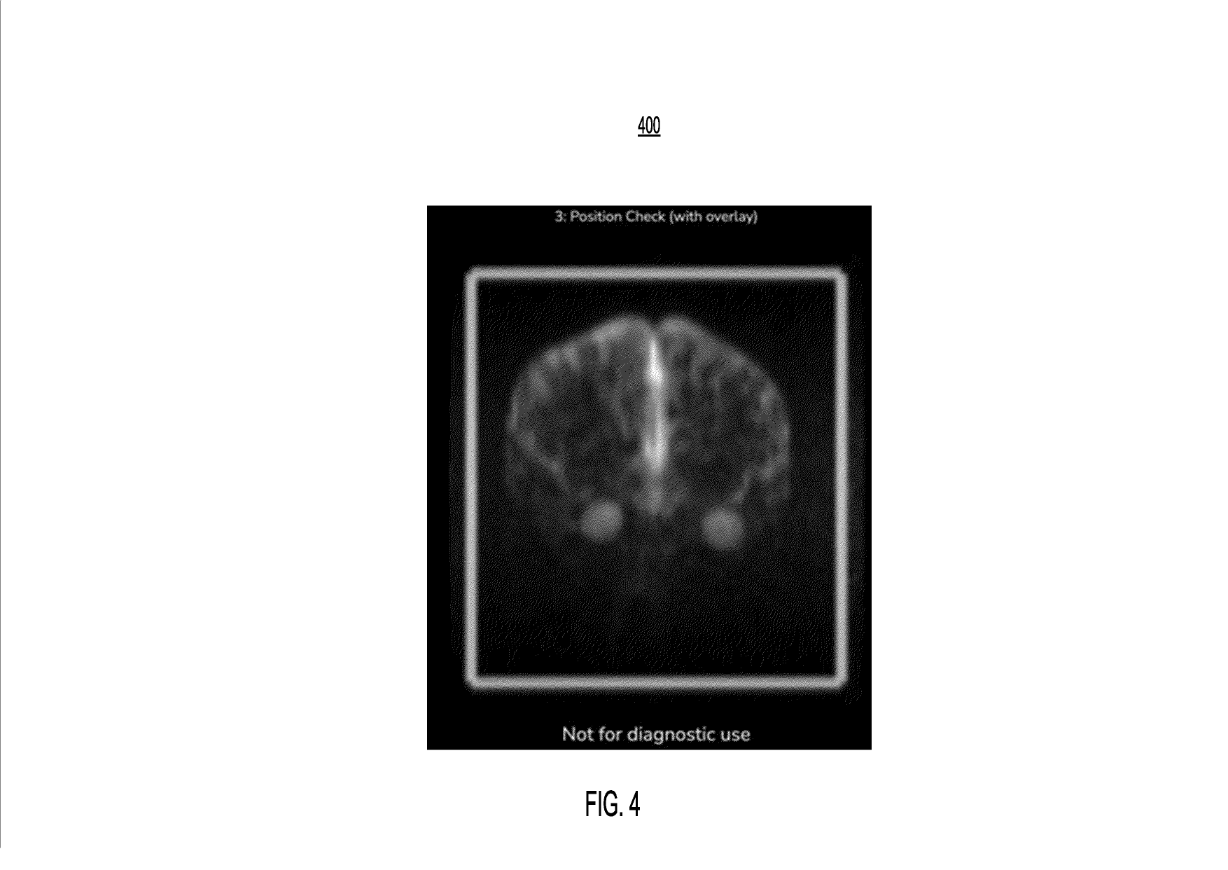

Referring to FIG. 4, illustrated is an example image generated using the techniques described herein including an indicator for positioning a subject, in accordance with one or more implementations. As shown, the indicator may be an overlay that indicates an imageable area or FOV of the MR system. In this example, the head of a subject is positioned correctly within the system (e.g., within a threshold distance of the RF coils), and therefore the overlay is a green bounding box. If the patient was not correctly positioned, the bounding box may be displayed in different colors (e.g., red, orange), as described herein, to reflect the amount/extent/distance by which the subject is to be moved to be aligned with the RF coils of the MR system.

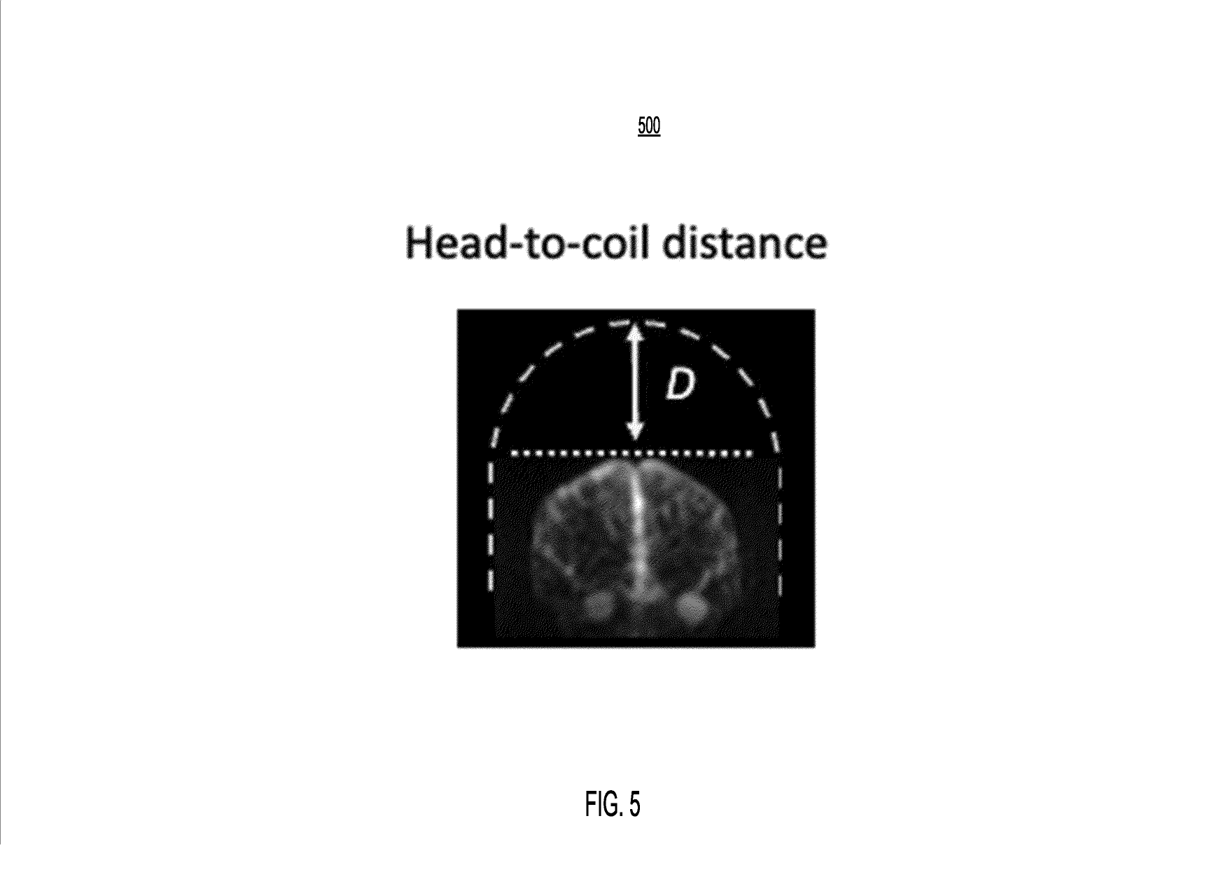

Referring to FIG. 5, illustrated is another example image generated using the techniques described herein, showing an amount by which a subject is misaligned with the MR system, in accordance with one or more implementations. In this example, the semicircular portion of the image represents a position of one or more RF coils of the MR system, and the distance D is the distance between the top of the region of interest of the subject (the subject’s brain, in this example).

Referring to FIG. 7, illustrated is an example position check image displayed in connection with an indicator, in accordance with one or more implementations. As described herein, multiple position check images may be captured at one time from different orientations or angles. In this example, the position check image is captured as depicting the top of the subject’s head. The green bounding box surrounding the imaged portion of the subject’s head represents an imageable area of the MR system. The bounding box (indicator) is green, indicating that the subject is correctly positioned within the imageable area. The distances D from each edge of the imageable area to the outer edges of the subject’s anatomy may be utilized by the MR system to determine whether the subject is correctly positioned within the MR system, as described herein.

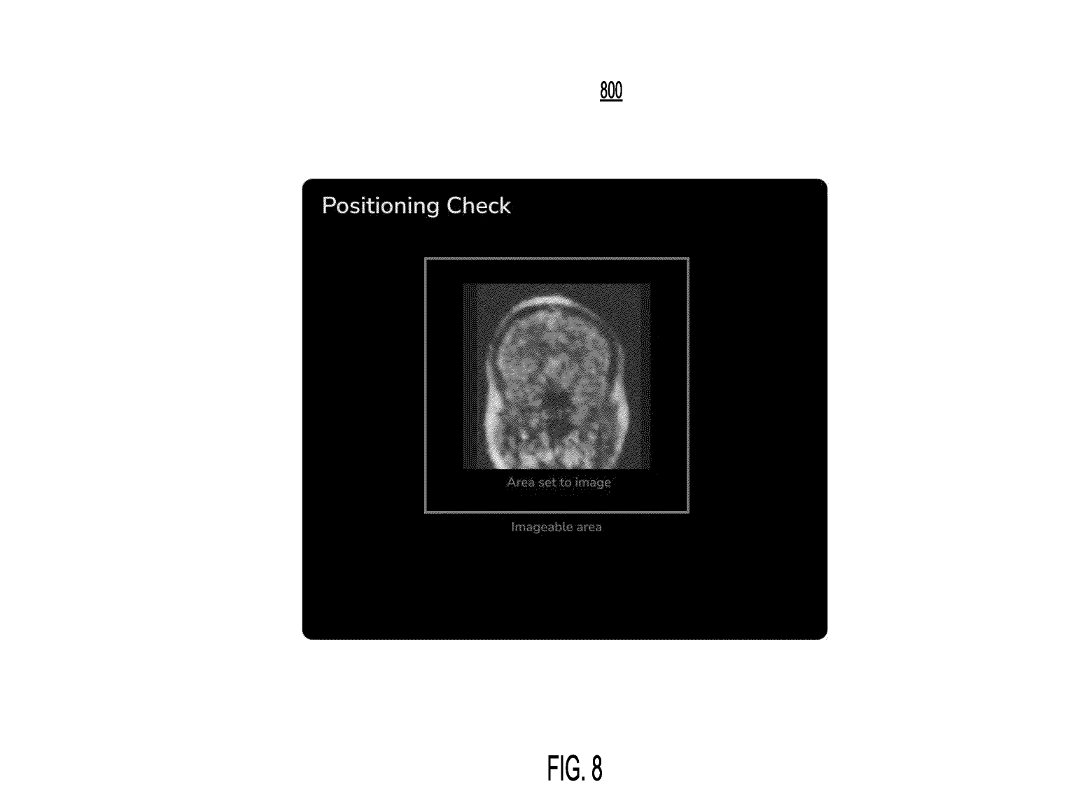

Referring to FIG. 8, illustrated is an example position check image displayed in connection with an overlay indicator (e.g., of any defined shape, size, contour, color, brightness. pattern, animation), in accordance with one or more implementations. In this example, the area set to image is represented by a rectangular overlay (e.g., an indicator). The rectangular overlay can represent the area set to be imaged by the MR system, which may be configurable by the operator of the MR system. Also as shown, an outer bounding box defines the imageable area of the MR system (e.g., the largest area that can be imaged). In this example, the head of the subject is positioned correctly within the boundaries of the imageable area or FOV, and therefore the overlay rectangle, representing the area to be imaged, is green.

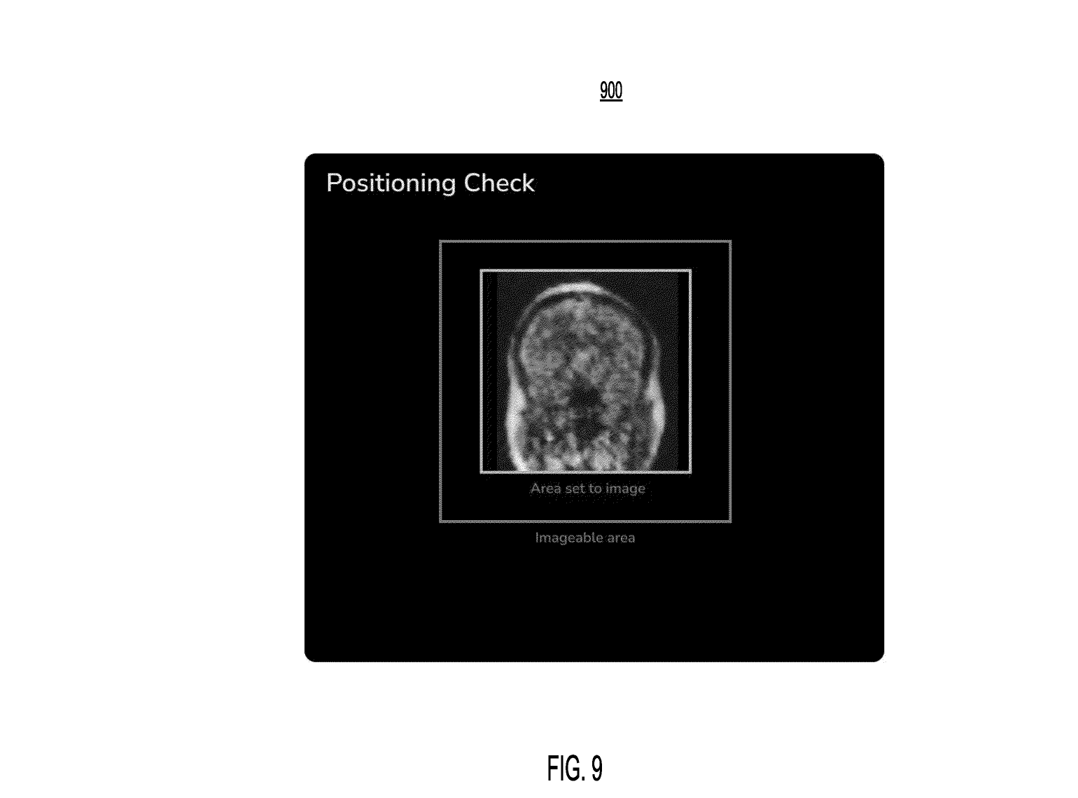

Referring to FIG. 9, illustrated is an example position check image displayed in connection with bounding box indicators for an area to be imaged and an imageable area, in accordance with one or more implementations. In this example, the area set to image is represented by a first green rectangular bounding box (e.g., an indicator). The first rectangular bounding box can represent the area set to be imaged by the MR system, which may be configurable by the operator of the MR system. Also as shown, a second, outer bounding box defines the imageable area of the MR system (e.g., the largest area that can be imaged). In this example, the head of the subject is positioned correctly within the boundaries of the imageable area, and therefore the first rectangular bounding box, representing the area to be imaged and surrounding the imaged region of the subject, is green.

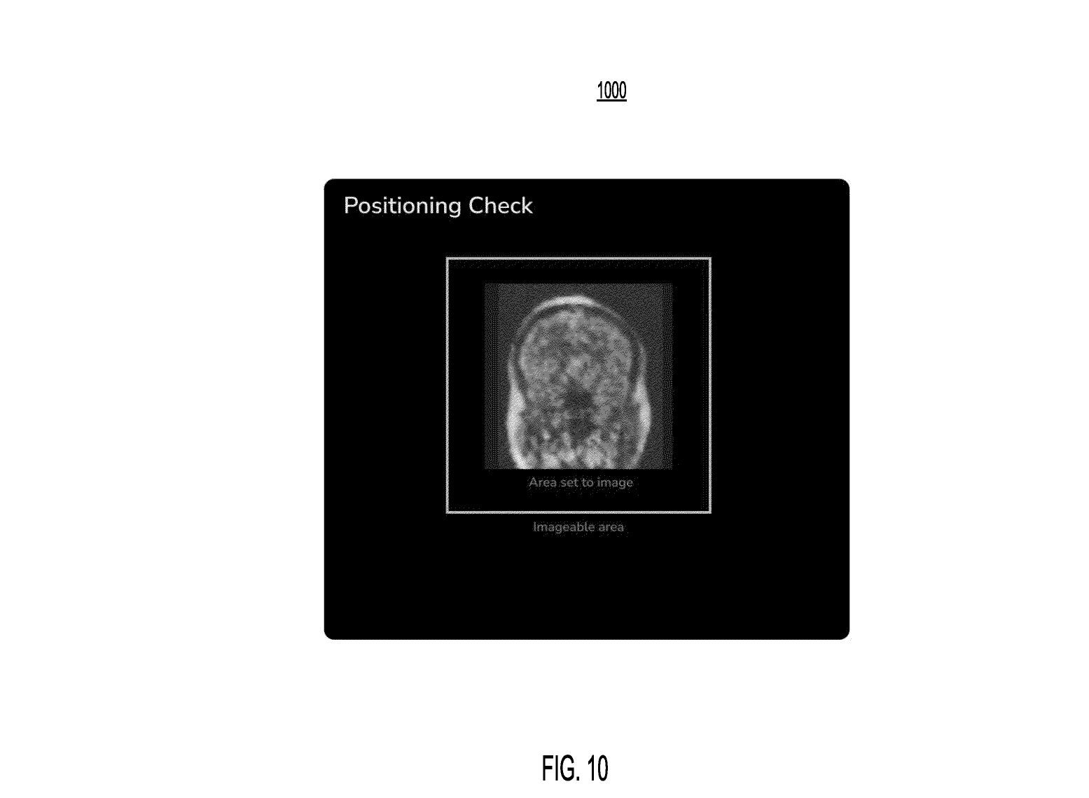

FIG. 10 illustrates another example position check image displayed in connection with an overlay indicator displayed within an area to be imaged and an imageable area, in accordance with one or more implementations. In this example, the area set to image is represented by a rectangular overlay (e.g., an indicator). The rectangular overlay can represent the area set to be imaged by the MR system, which may be configurable by the operator of the MR system. Also as shown, an outer, green bounding box defines the imageable area of the MR system (e.g., the largest area that can be imaged). In this example, the head of the subject is positioned correctly within the boundaries of the imageable area, and therefore both the bounding both the overlay rectangle and the outer bounding box representing the imageable area are green.

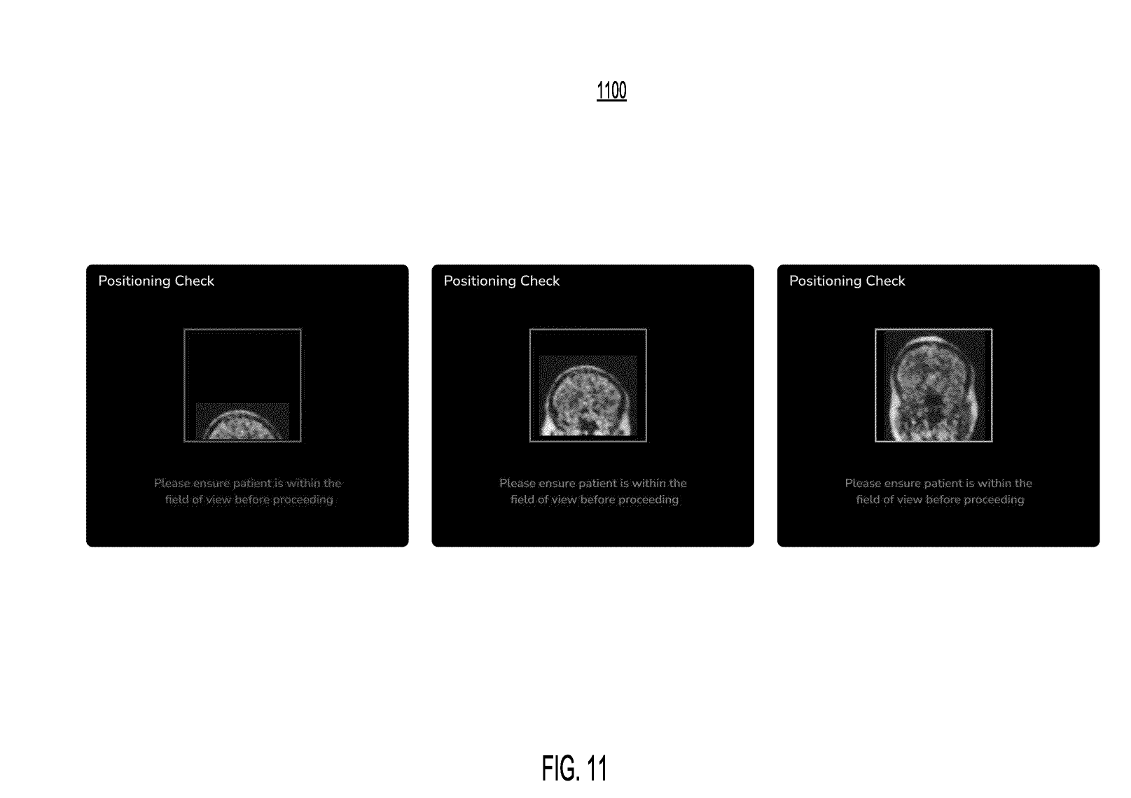

Referring to FIG. 11, illustrated are side-by-side comparisons of position check images displayed in connection with corresponding overlay indicators at various stages of alignment with the MR system, in accordance with one or more implementations. As shown, the subject depicted in the left-most image is the most out-of-frame relative to the other images shown in FIG. 13, and therefore the bounding box representing the area set to image is red. As the subject is repositioned closer to the optimal position for imaging within the MR system, the MR system modifies the indicator to be orange, as shown in the middle image. Once the subject has been correctly positioned within the MR system, the bounding box indicator can be shown in green, as shown in the right-most position check image.

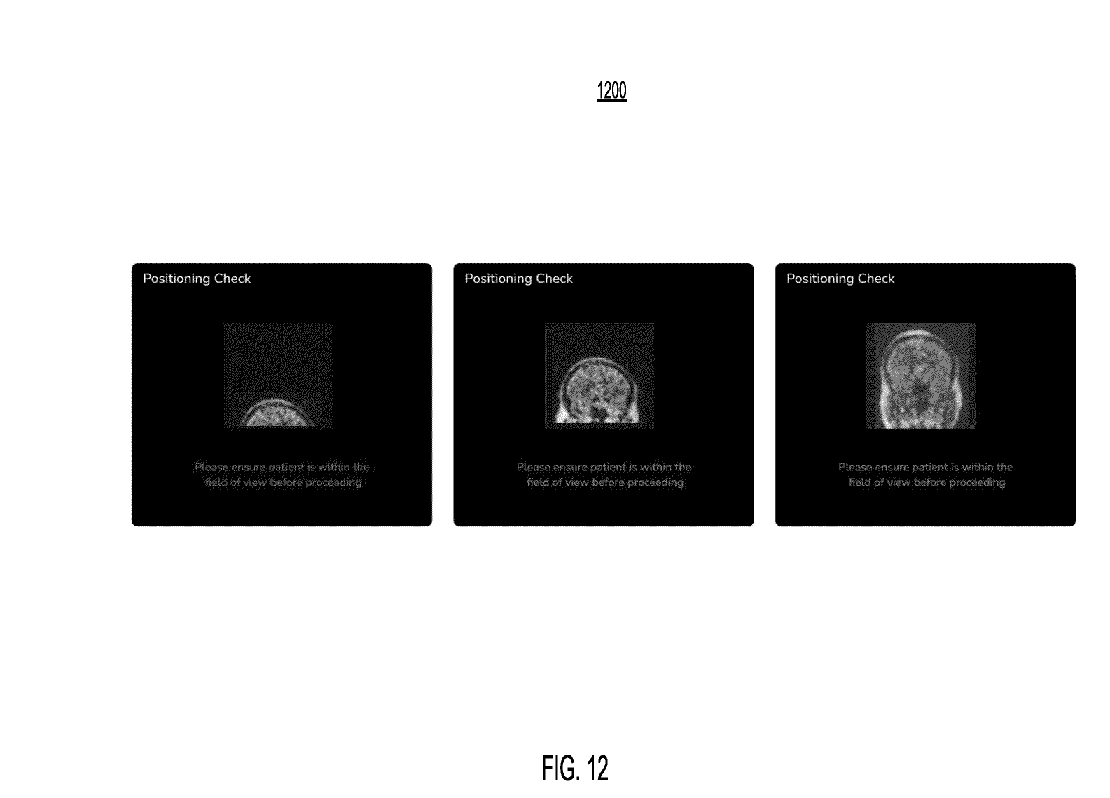

Referring to FIG. 12, illustrated are side-by-side comparisons of position check images displayed in connection with corresponding overlay indicators at various stages of alignment with the MR system, in accordance with one or more implementations. As shown, the subject depicted in the left-most image is the most out-of-frame relative to the other images shown in FIG. 13, and therefore the overlay indicator representing the area set to image is red. As the subject is repositioned closer to the optimal position for imaging within the MR system, the MR system modifies the overlay indicator to be orange, as shown in the middle image. Once the subject has been correctly positioned within the MR system, the overlay indicator can be presented in a green color, as shown in the right-most position check image.

FIGS. 13A – 13C and 14 illustrate various example embodiments of the disclosed positioning system. The live patient positioning feature enables the user to see visual feedback in the device’s user interface of the patient’s position in comparison to the field of view. By showing the user visual feedback live, the user is able to reposition and adjust the patient to optimize the position faster. As depicted, the visualization is on the same tab as the exam list so the user does not need to switch between tabs or pages to see the image. In example embodiments, the live patient positing feature allows the user to see the position of a patient as the patient moves into the system, as opposed to needing to stop to check the feedback and then readjust. This workflow feature allows for a substantial time savings and optimal use of hospital staffing resources. This improvement encourages the user to be looking at the visual by putting the visual on the same tab as the exam list. Checking patient position in the user interface without the feedback being live might require the user to potentially go back and forth between positioning and verifying. Various embodiments further improve the user experience by condensing the exam and the view tabs so the user is not flipping between these tabs to verify the patient’s position in the field of view. The system provides improved patient positioning, improved speed to position the patient, and optimized use of staffing resources needed to load the patient.

In various embodiments, a live positioning pulse sequence is based on a single shot fast spin echo acquisition which is repeated in a loop until it is stopped. In example embodiments, 3D volume is excited, but 2D image is acquired. This represents projection in the second phase-encode dimension. In other words, if imaging is performed in coronal scan plane, the resulting image would look like all coronal slices of the head were summed together. The time between repetitions is controlled by a parameter called repetition time. This method can be performed in any scan plane, with or without fat suppression. In example embodiments, each acquisition and reconstruction may be performed in less than a second.

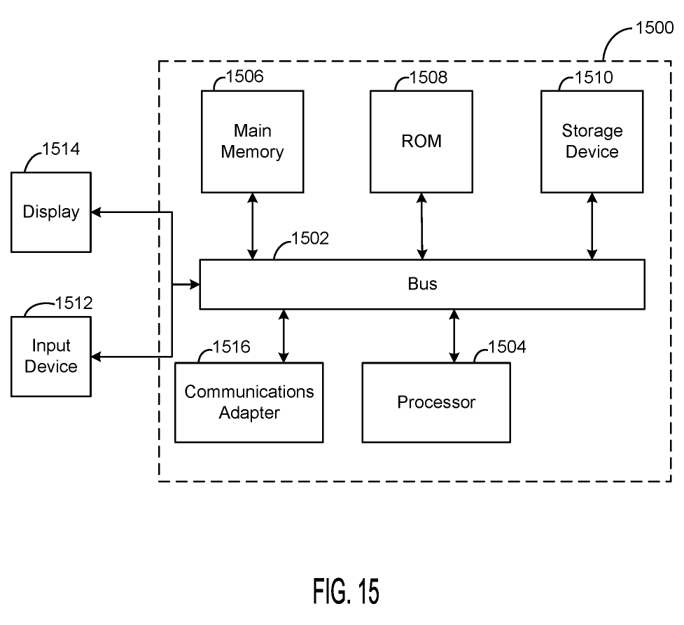

FIG. 15 illustrates a component diagram of an example computing system suitable for use in the various implementations described herein, according to an example implementation. In a non-limiting example, the computing system 1500 may implement a computing device 104 or controller 106 of FIG. 1, or various other example systems and devices described in the present disclosure.

The computing system 1500 includes a bus 1002 or other communication component for communicating information and a processor 1504 coupled to the bus 1502 for processing information. The computing system 1500 also includes main memory 1506, such as a RAM or other dynamic storage device, coupled to the bus 1502 for storing information, and instructions to be executed by the processor 1504. Main memory 1506 may also be used for storing position information, temporary variables, or other intermediate information during execution of instructions by the processor 1504. The computing system 1500 may further include a ROM 1508 or other static storage device coupled to the bus 1502 for storing static information and instructions for the processor 1504. A storage device 1510, such as a solid-state device, magnetic disk, or optical disk, is coupled to the bus 1502 for persistently storing information and instructions.

The computing system 1500 may be coupled via the bus 1502 to a display 1514, such as a liquid crystal display, or active matrix display, for displaying information to a user. An input device 1512, such as a keyboard including alphanumeric and other keys, may be coupled to the bus 1502 for communicating information, and command selections to the processor 1504. In another implementation, the input device 1512 has a touch screen display. The input device 1512 may include any type of biometric sensor, or a cursor control, such as a mouse, a trackball, or cursor direction keys, for communicating direction information and command selections to the processor 1504 and for controlling cursor movement on the display 1514.

In some implementations, the computing system 1500 may include a communications adapter 1516, such as a networking adapter. Communications adapter 1516 may be coupled to bus 1502 and may be configured to enable communications with a computing or communications network or other computing systems. In various illustrative implementations, any type of networking configuration may be achieved using communications adapter 1516, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein may be achieved by the computing system 1500 in response to the processor 1504 executing an implementation of instructions contained in main memory 1506. Such instructions may be read into main memory 1506 from another computer-readable medium, such as the storage device 1510. Execution of the implementation of instructions contained in main memory 1506 causes the computing system 1500 to perform the illustrative processes described herein. One or more processors in a multi-processing implementation may also be employed to execute the instructions contained in main memory 1506. In alternative implementations, hard-wired circuitry may be used in place of or in combination with software instructions to implement illustrative implementations. Thus, implementations are not limited to any specific combination of hardware circuitry and software.

The implementations described herein have been described with reference to drawings. The drawings illustrate certain details of specific implementations that implement the systems, methods, and programs described herein. Describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. In a non-limiting example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, in a non-limiting example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, in a non-limiting example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. In a non-limiting example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

Various non-limiting example embodiments include:

Embodiment A1: A method comprising: capturing preliminary imaging data indicative of a first position of a subject in an imaging system; making a determination regarding the first position of the subject based on the preliminary imaging data; and capturing subsequent imaging data of the subject based on the determination.

Embodiment A2: The method of Embodiment A1, wherein the preliminary imaging data is captured to position the subject in an imaging space or field of view (FOV) of the imaging system, and the subsequent imaging data is captured to image a region of interest (ROI) of the subject.

Embodiment A3: The method of Embodiment A2, wherein the preliminary imaging data is captured in no more than about 10 seconds, and the subsequent imaging data requires more than 10 seconds to be captured.

Embodiment A4: The method of Embodiment A2, wherein the preliminary imaging data is captured and one or more preliminary images generated based on the preliminary imaging data, the images generated in no more than about 10 seconds.

Embodiment A5: The method of Embodiment A2, wherein the preliminary imaging data is captured in no more than about 5 seconds, and the subsequent imaging data requires more than 5 seconds to be captured.

Embodiment A6: The method of Embodiment A2, wherein the preliminary imaging data is captured and one or more preliminary images generated based on the preliminary imaging data in no more than about 5 seconds.

Embodiment A7: The method of Embodiment A2, wherein the preliminary imaging data is captured in no more than about 3 seconds, and the subsequent imaging data requires more than 30 seconds to be captured.

Embodiment A8: The method of Embodiment A2, wherein the preliminary imaging data is captured and one or more preliminary images generated based on the preliminary imaging data, the images generated in no more than about 3 seconds.

Embodiment A9: The method of any of Embodiments A1 – A8, wherein the preliminary imaging data is first preliminary imaging data, wherein the determination is that the subject is to be moved prior to capturing the subsequent imaging data, and wherein the subject is moved to a second position prior to capturing the subsequent imaging data.

Embodiment A10: The method of any of Embodiments A1 – A9, wherein additional preliminary imaging data is captured and the subject iteratively moved based on the additional preliminary imaging data until the subject is in a desired position for imaging prior to capturing the subsequent imaging data.

Embodiment A11: The method of any of Embodiments A1 – A10, wherein the imaging system is a magnetic resonance (MR) system.

Embodiment A12: The method of any of Embodiments A1 – A11, wherein the MR system is a low-field MR imaging system.

Embodiment A13: The method of any of Embodiments A1 – A12, wherein the MR system is a portable MR imaging system.

Embodiment A14: The method of any of Embodiments A1 – A13, wherein the MR imaging system uses on average no more than 5 kilowatts during operation.

Embodiment A15: The method of any of Embodiments A1 – A14, wherein the MR imaging system comprises a motor and a battery for self-propelled movement.

Embodiment A16: The method of any of Embodiments A1 – A15, wherein the preliminary imaging data comprises two-dimensional (2D) imaging data.

Embodiment A17: The method of any of Embodiments A1 – A16, wherein the preliminary imaging data consists of 2D imaging data.

Embodiment A18: The method of any of Embodiments A1 – A17, wherein the preliminary imaging data comprises one-dimensional (1D) imaging data.

Embodiment A19: The method of any of Embodiments A1 – A18, wherein the preliminary imaging data consists of 1D imaging data.

Embodiment A20: The method of any of Embodiments A1 – A19, further comprising generating one or more preliminary images based on the preliminary imaging data.

Embodiment A21: The method of Embodiment A20, wherein generating the one or more preliminary images comprises projecting 3D imaging data into two dimensions.

Embodiment A22: The method of Embodiment A20, further comprising displaying the one or more preliminary images.

Embodiment A23: The method of any of Embodiments A1 – A22, further comprising displaying a visualization based on the preliminary imaging data.

Embodiment A24: The method of Embodiment A23, wherein the visualization comprises a representation of a boundary or perimeter of an imaging space of the imaging system.

Embodiment A25: The method of Embodiment A24, wherein the visualization further comprises an indication of a distance from the boundary or perimeter to a body part of the subject.

Embodiment A26: The method of Embodiment A23, wherein the visualization comprises a representation of a boundary or perimeter of a FOV of the imaging system.

Embodiment A27: The method of Embodiment A26, wherein the visualization further comprises an indication of a distance from the boundary or perimeter to a body part of the subject.

Embodiment A28: The method of Embodiment A24, wherein the visualization comprises a representation of a boundary or perimeter of a component of the imaging system.

Embodiment A29: The method of Embodiment A28, wherein the visualization further comprises an indication of a distance from the boundary or perimeter to a body part of the subject.

Embodiment A30: The method of Embodiment A23, wherein the visualization comprises a representation of a boundary or perimeter of a body part of the subject.

Embodiment A31: The method of any of Embodiments A1 – A30, wherein the preliminary imaging data corresponds to a set of sequential images captured prior to capturing the subsequent imaging data.

Embodiment A32: The method of Embodiment A31, further comprising displaying the set of sequential images on a display screen to aid positioning of the subject.

Embodiment A33: The method of Embodiment A32, wherein images are displayed from more than one perspective.

Embodiment A34: The method of Embodiment A32, wherein the set of sequential images has an average frame rate of at least about 0.5 images per second.

Embodiment A35: The method of Embodiment A34, wherein the set of sequential images has an average frame rate of at least about 1 image per second.

Embodiment A36: The method of Embodiment A32, wherein the set of sequential images has an average frame rate of at least about 2 images per second.

Embodiment A37: The method of Embodiment A32, wherein the set of sequential images are displayed with an overlay.

Embodiment A38: The method of Embodiment A37, wherein the overlay corresponds to a FOV of the imaging system.

Embodiment A39: The method of Embodiment A37, wherein the overlay corresponds to a component of the imaging system.

Embodiment A40: The method of any of Embodiments A1 – A39, further comprising providing an indication of the first position or a second position of the subject.

Embodiment A41: The method of Embodiment A40, wherein the indication comprises an audible signal.

Embodiment A42: The method of Embodiment A40, wherein the indication comprises a visual indicator.

Embodiment A43: The method of Embodiment A42, wherein the indication further comprises an audible indicator.

Embodiment A44: The method of Embodiments A42, wherein the visual indicator comprises a change in color in a visualization displayed on a screen.

Embodiment A45: The method of Embodiment A44, wherein the change is from a first color to a second color indicative of the second position of the subject.

Embodiment A46: The method of Embodiment A42, wherein the visual indicator comprises a prompt displayed on a screen.

Embodiment A47: The method of Embodiment A46, wherein the prompt comprises guidance on positioning or repositioning the subject.

Embodiment A48: The method of Embodiment A46, wherein the prompt comprises a selectable icon for instructing the imaging system to capture additional preliminary imaging data to identify a current position of the position.

Embodiment A49: The method of Embodiment A48, wherein the visual indicator comprises one or more lights turning on and/or off.

Embodiment A50: The method of any of Embodiments A1 – A49, further comprising repositioning the subject to a second position based on the determination, wherein the subsequent image of the subject follows the repositioning of the subject.

Embodiment A51: The method of any of Embodiments A1 – A50, wherein the preliminary imaging data is captured using an imaging device configured to detect visible light.

Embodiment A52: The method of any of Embodiments A1 – A51, wherein the preliminary imaging data is captured following acquisition of calibration data for the subject.

Embodiment A53: The method of any of Embodiments A1 – A52, wherein making the determination comprises computing a distance from a body part of the subject to a component of the imaging system.

Embodiment A54: The method of Embodiment A52, wherein the method comprises providing a warning if the distance is greater than a threshold.

Embodiment A55: The method of any of Embodiments A1 – A54, further comprising making a second determination that the subject has deviated from a desirable position following a start of the capturing of the subsequent imaging data.

Embodiment B1: A method comprising: capturing preliminary positioning data indicative of a first position of a subject in an imaging system; making a determination regarding the first position of the subject based on the preliminary positioning data; and capturing imaging data corresponding to a ROI of the subject.

Embodiment B2: The method of Embodiment B1, wherein the preliminary positioning data comprises imaging data.

Embodiment B3: The method of either Embodiment B1 or B2, wherein the preliminary positioning data comprises sensor data.

Embodiment C1: An imaging system configured to perform any of the above methods.

Embodiment C2: The imaging system of Embodiment C1, wherein the imaging system is an MRI system.

Embodiment C3: The imaging system of either Embodiment C1 or C2, wherein the MRI system is a low-field MRI system.

Embodiment C4: The imaging system of any of Embodiments C1 – C3, wherein the MRI system is a portable MRI system.

Embodiment C5: The imaging system of any of Embodiments C1 – C4, wherein the MRI system comprises a camera for acquiring images of the subject during positioning of the subject.

Embodiment C6: The imaging system of any of Embodiments C1 – C5, further comprising one or more sensors for acquiring sensor data indicative of the subject’s position.

Embodiment D1: A method comprising: capturing preliminary imaging data indicative of a first position of a subject in a portable magnetic resonance imaging (MRI) system; making a determination regarding the first position of the subject based on the preliminary imaging data; capturing subsequent imaging data of the subject based on the determination; and providing image data to a user of the MRI system, wherein the image data comprises, or is derived from, at least one of the preliminary imaging data or the subsequent imaging data, and wherein providing the image data to the user comprises at least one of: (i) transmitting the image data to a computing system, and/or (ii) displaying a visualization based on the image data on a display screen.

Embodiment D2: The method of Embodiment D1, wherein the preliminary imaging data is captured to position the subject in an imaging space or field of view (FOV) of the imaging system, and the subsequent imaging data is captured to image a region of interest (ROI) of the subject.

Embodiment D3: The method of either Embodiment D1 or D2, wherein the portable MR system is a low-field MR imaging system.

Embodiment D4: The method of any of Embodiments D1 – D3, wherein the MR imaging system comprises a motor and a battery for self-propelled movement.

Embodiment D5: The method of any of Embodiments D1 – D4, wherein the preliminary imaging data comprises two-dimensional (2D) imaging data.

Embodiment D6: The method of any of Embodiments D1 – D5, further comprising displaying one or more images based on the image data.

Embodiment D7: The method of any of Embodiments D1 – D6, further comprising displaying a visualization based on the image data.

Embodiment D8: The method of Embodiment D7, wherein the visualization further comprises an indication of a distance from a boundary or perimeter to a body part of the subject.

Embodiment D9: The method of Embodiment D7, wherein the visualization comprises a representation of a boundary or perimeter of a component of the imaging system.

Embodiment D10: The method of any of Embodiments D1 – D9, further comprising displaying a set of sequential images on the display screen to aid positioning of the subject, the set of sequential images being based on the preliminary imaging data captured prior to capturing the subsequent imaging data.

Embodiment D11: The method of Embodiment D11, wherein the set of sequential images are displayed with an overlay.

Embodiment D12: The method of any of Embodiments D1 – D11, further comprising providing one or more prompts with guidance on positioning or repositioning the subject.

Embodiment D13: The method of any of Embodiments D1 – D12, wherein image data is transmitted to a mobile computing device with a display.

Embodiment D14: The method of Embodiment D13, wherein the mobile computing device is a tablet, a smartphone, a laptop computer, or a personal digital assistant.

Embodiment D15: The method of any of Embodiments D1 – D14, wherein image data is transmitted to a workstation or desktop computing system.

Embodiment D16: The method of any of Embodiments D1 – D15, wherein image data is transmitted to at least one computing system and at least one mobile computing device.

Embodiment E1: A portable magnetic resonance imaging (MRI) system comprising one or more processors, the portable MRI system configured to: capture, in response to a first user input, preliminary imaging data indicative of a first position of a subject in the portable MRI system; capture, in response to a second user input, subsequent imaging data of the subject; and provide image data to a user of the MRI system, wherein the image data comprises, or is derived from, at least one of the preliminary imaging data or the subsequent imaging data, and wherein providing the image data to the user comprises at least one of: (i) transmitting the image data to a computing system, and/or (ii) displaying a visualization based on the image data on a display screen.

Embodiment E2: The MRI system of Embodiments E1, wherein image data is transmitted to a mobile computing device with a display.

Embodiment E3: The MRI of Embodiment E2, wherein the mobile computing device is a tablet, a smartphone, a laptop computer, or a personal digital assistant.

Embodiment E4: The MRI system of any of Embodiments E1 – E3, wherein image data is transmitted to a workstation or desktop computing system.

Embodiment E5: The MRI system of any of Embodiments E1 – E4, wherein image data is transmitted to at least one computing system and at least one mobile computing device.

Embodiment E6: The method of any of Embodiments E1 – E5, wherein the preliminary imaging data captured by the MRI system is not for generation of an image usable for a diagnosis of a disease or condition, and wherein the subsequent imaging data is for generation of an image that is usable for the diagnosis of the disease or condition.

Embodiment F1: A method, system, or a non-transitory computer-readable storage medium, wherein the preliminary imaging data is captured using a pulse sequence comprising a single-shot fast spin echo acquisition repeated in a loop until terminated, the acquisition exciting a three-dimensional volume and acquiring a two-dimensional projection image representing a summation of slices in a second phase-encode dimension.

Embodiment F2: A method, system, or a non-transitory computer-readable storage medium, wherein the visualization comprises a color-coded indicator that changes color based on a degree of alignment of the subject with a field of view of the MRI system, the colors including at least a first color indicating misalignment, a second color indicating partial alignment, and a third color indicating proper alignment.

Embodiment F3: A method, system, or a non-transitory computer-readable storage medium, wherein the determination regarding the first position of the subject is performed by a trained machine learning model configured to receive the preliminary imaging data as input and output an estimated distance between a body part of the subject and a component of the MRI system.

Embodiment F4: A method, system, or a non-transitory computer-readable storage medium, further comprising a motorized transport system configured to detect a force applied to the MRI system and, in response, provide motorized assistance in a direction of the applied force to reposition the MRI system.

Embodiment F5: A method, system, or a non-transitory computer-readable storage medium, wherein the preliminary imaging data is captured periodically at a rate of at least one image per second while the subject is being moved into the MRI system, and wherein the visualization is updated in near real time on a same display tab as an exam list to reduce operator workflow steps.

Embodiment G1: A non-transitory computer-readable storage medium comprising instructions that, when executed by one or more processors of a computing system and/or computing device, cause the computing system and/or computing device to perform any of the methods of Embodiments A1 – A55, B1 – B3, D1 – D16, or F1 – F5.

Embodiment H1: A computing system and/or computing device comprising any combination of the non-transitory computer-readable storage media of Embodiment G1.

What is claimed is:

1. A method comprising:
capturing preliminary imaging data indicative of a first position of a subject in a portable magnetic resonance imaging (MRI) system;
making a determination regarding the first position of the subject based on the preliminary imaging data;
capturing subsequent imaging data of the subject based on the determination; and
providing image data to a user of the MRI system, wherein the image data comprises, or is derived from, at least one of the preliminary imaging data or the subsequent imaging data, and wherein providing the image data to the user comprises at least one of:
(i) transmitting the image data to a computing system, and/or
(ii) displaying a visualization based on the image data on a display screen.

2. The method of claim 1, wherein the preliminary imaging data is captured to position the subject in an imaging space or field of view (FOV) of the imaging system, and the subsequent imaging data is captured to image a region of interest (ROI) of the subject.

3. The method of claim 1, wherein the portable MR system is a low-field MR imaging system.

4. The method of claim 1, wherein the MR imaging system comprises a motor and a battery for self-propelled movement.

5. The method of claim 1, wherein the preliminary imaging data comprises two-dimensional (2D) imaging data.

6. The method of claim 1, further comprising displaying one or more images based on the image data.

7. The method of claim 1, further comprising displaying a visualization based on the image data.

8. The method of claim 7, wherein the visualization further comprises an indication of a distance from a boundary or perimeter to a body part of the subject.

9. The method of claim 7, wherein the visualization comprises a representation of a boundary or perimeter of a component of the imaging system.

10. The method of claim 1, further comprising displaying a set of sequential images on the display screen to aid positioning of the subject, the set of sequential images being based on the preliminary imaging data captured prior to capturing the subsequent imaging data.

11. The method of claim 10, wherein the set of sequential images are displayed with an overlay.

12. The method of claim 1, further comprising providing one or more prompts with guidance on positioning or repositioning the subject.

13. The method of claim 1, wherein image data is transmitted to a mobile computing device with a display.

14. A portable magnetic resonance imaging (MRI) system comprising one or more processors, the portable MRI system configured to:
capture, in response to a first user input, preliminary imaging data indicative of a first position of a subject in the portable MRI system;
capture, in response to a second user input, subsequent imaging data of the subject; and
provide image data to a user of the MRI system, wherein the image data comprises, or is derived from, at least one of the preliminary imaging data or the subsequent imaging data, and wherein providing the image data to the user comprises at least one of:
(i) transmitting the image data to a computing system, and/or
(ii) displaying a visualization based on the image data on a display screen.

15. The portable MRI system of claim 14, wherein the preliminary imaging data captured by the MRI system is not for generation of an image usable for a diagnosis of a disease or condition, and wherein the subsequent imaging data is for generation of an image that is usable for the diagnosis of the disease or condition.
